(19) United States
(12) Patent Application Publication
(10) Pub. No.: US 2012/0330339 A1
(43) Pub. Date: Dec. 27, 2012

(54) TISSUE CUTTING DEVICE, ASSEMBLY AND METHOD

(75) Inventor: Mehmet Ziya Sengun, Raynham, MA (US)

(73) Assignee: DePuy Mitek, Inc., Raynham, MA (US)

(21) Appl. No.: 13/166,135

(22) Filed: Jun. 22, 2011

(51) Int. Cl. A61B 17/3205; A61B 17/34

(52) U.S. Cl. 606/170

(57) ABSTRACT

A tissue cutting device, assembly and method of using same, including a generally cylindrical tube having a distal end and a proximal end and defining an open passage extending from the proximal end to the distal end. The distal end defines a cutting edge, and the tube has a substantially smooth exterior along a working length selected for insertion through the epidermis of a patient to access tissue to be cut. The assembly further includes a grasping tool having a distal portion defining a grasping element capable of grasping tissue and of being inserted through the passage of the tube. The tube and the grasping tool are separable and independent from each other, and the tube is advanceable relative to the tool to cut tissue held by the grasping element.

### BACKGROUND OF THE INVENTION

1. Field of the Invention

The invention relates to devices for cutting tissue in a patient utilizing a tube having a cutting edge.

2. Description of the Related Art

There are a number of procedures requiring tissue to be cut and removed from a patient. One such procedure is a meniscectomy during which a portion of a torn or otherwise damaged meniscus is cut and removed arthroscopically. Other procedures for shoulder repair typically utilize a first cannula to pass an arthroscope for visualization and at least a second cannula to admit a cutting tool such as a shaver or morcellator. Saline is injected continuously to expand the joint tissues and to clean debris. It can be difficult to position and hold tissue during cutting, and removal of large quantities of tissue can be time-consuming.

Arthroscopic sheaths for knee surgery are typically at least three millimeters in diameter and three to five inches in length. Cannulas for shoulder repair have the same or larger diameter, and are typically four to six inches in length. Sutures are usually utilized to close wounds created during insertion of sheaths or cannulas having a diameter of three millimeters or more. Smaller cannula with low-profile graspers, shavers and morcellators have been used for hand and ear-nose-throat surgeries.

One example of a cutting and grasping device with three concentric tubes, each with a side window, is provided in U.S. Pat. No. 5,947,983 by Solar et al. The side windows are alignable with each other such that two of the tubes grasp tissue while the third tube cuts the tissue. Only tissue within the windows is cut.

Another known cutting device is a laparoscopic morcellator disclosed by Savage et al. in U.S. Pat. No. 6,039,748. A rotating tubular cutting member is mechanically driven within an outer tube. An inner tube is rotationally fixed and axially movable relative to the tubular cutting member. An endoscopic grasper may be inserted through the inner tube having a valve to minimize loss of insufflations gas.

Other cutting devices are described by Baxter et al. in U.S. Pat. No. 7,033,357 and by Pravong et al. in U.S. Patent Publication No. 2008/0255597, for example.

Dermal biopsy punches are an example of topical cutting devices. Biopsy punches manufactured by Miltex, Inc. of York, Pa., have a relatively short cylindrical tube, less than one centimeter in length, with a distal annular cutting edge. The tube is carried by an elongated polymeric cylindrical grip to facilitate manual rotation of the tube.

It is therefore desirable to have a low-cost, minimally invasive device for cutting tissue within a patient that easily enables grasping of the tissue during cutting.

### SUMMARY OF THE INVENTION

An object of the present invention is to provide a simple, low-profile cutting device which does not require a separate cannula to access tissue within a patient.

Another object of the present invention is to increase the number of possible insertion sites at an area of the patient to be treated.

A still further object of the present invention is to obviate the need for sutures to close a channel created during insertion of such a cutting device.

Yet another object of the present invention is to provide such a cutting device which is not coupled to a specific grasping tool, thereby allowing a desired low-profile grasper to be utilized with the cutting device.

This invention features a tissue cutting assembly including a generally cylindrical tube having a distal end and a proximal end and defining an open passage extending from the proximal end to the distal end. The distal end defines a cutting edge, and the tube has a substantially smooth exterior along a working length selected for insertion through the epidermis of a patient to access tissue to be cut. The assembly further includes a grasping tool having a distal portion defining a grasping element capable of grasping tissue and of being inserted through the passage of the tube. The tube and the grasping tool are separable and independent from each other, and the tube is advanceable relative to the tool to cut tissue held by the grasping element.

In some embodiments, the cutting edge is substantially annular, and is smooth in one embodiment and serrated in another embodiment. In certain embodiments, at least a portion of the tube is capable of being bent during insertion of the grasping element, such as by having at least one cut-out formed in the tube.

In certain embodiments, the proximal portion of the tube includes an enhanced gripping surface such as a textured or knurled surface in some embodiments and a polymeric coating or molded overlay in other embodiments. Preferably, the working length of the tube has an outer diameter of less than three millimeters, more preferably two millimeters or less, and a length of at least three centimeters.

This invention also features a method of cutting tissue by inserting into a patient a generally cylindrical tube having a distal end and a proximal end and defining an open passage extending from the proximal end to the distal end, with the distal end defining a cutting edge. The method further includes inserting into the passage a separate and independent grasping tool having a distal portion defining a grasping element capable of grasping tissue and of being inserted through the passage of the tube. A portion of tissue is grasped with the grasping element, and the tube is advanced to cut tissue around the grasped portion. The grasped portion of the tissue is then removed from the patient. In certain embodiments, the step of inserting the tube includes placing one of a trocar and an obturator in the passage of the tube to substantially block the passage, pushing the tube through the epidermis and underlying tissues until a desired depth is reached, and then removing the one of the trocar and the obturator before insertion of the grasping tool.

This invention may also be expressed as a tissue cutting device consisting essentially of a generally cylindrical metallic tube having a distal end and a proximal end and defining an open passage extending from the proximal end to the distal end, the distal end defining a cutting edge, and the tube having a substantially smooth exterior along a working length selected for insertion through the epidermis of a patient to access tissue to be cut. The working length of the tube has a length of at least three centimeters and has an outer diameter of less than three millimeters.

### BRIEF DESCRIPTION OF THE DRAWINGS

In what follows, preferred embodiments of the invention are explained in more detail with reference to the drawings, in which:

FIG. 1 is a schematic cross-sectional view of a cutting tube according to the present invention;

FIG. 2 is a schematic side cross-sectional view of an alternative cutting tube inserted through skin of a patient together with a grasping tool as an assembly according to the present invention;

FIG. 3 is a schematic top cross-sectional view of the tube of FIG. 2 together with an alternative grasping tool having a curved distal grasping element;

FIG. 4 is a schematic side view of the assembly of FIG. 2 with the grasping tool advanced to grasp tissue;

FIG. 5 is a schematic side view showing the tube being advanced to begin cutting tissue;

FIG. 6 is a schematic side view showing the tube cutting through tissue;

FIG. 7 illustrates the grasper after removal from the patient and from the tube with a strip of tissue;

FIG. 8A is a schematic side view of a hollow trocar utilizable according to the present invention;

FIG. 8B is a perspective view of the distal end of the trocar of FIG. 8A;

FIG. 9 is a schematic side view of an alternative solid trocar;

FIG. 10 is a schematic side view of the trocar of FIG. 9 inserted within a cutting tube according to the present invention; and

FIG. 11 is a schematic side view of a portion of a grasping element utilizable as an obturator according to the present invention.

### DETAILED DESCRIPTION OF THE PRESENTLY PREFERRED EMBODIMENTS

This invention may be accomplished by a tissue cutting assembly, and method of using same, including a generally cylindrical tube having a distal end and a proximal end and defining an open passage extending from the proximal end to the distal end. The distal end defines a cutting edge, and the tube has a substantially smooth exterior along a working length selected for insertion through the epidermis of a patient to access tissue to be cut. The assembly further includes a grasping tool having a distal portion defining a grasping element capable of grasping tissue and of being inserted through the passage of the tube. The tube and the grasping tool are separable and independent from each other, and the tube is advanceable relative to the tool to cut tissue held by the grasping element.

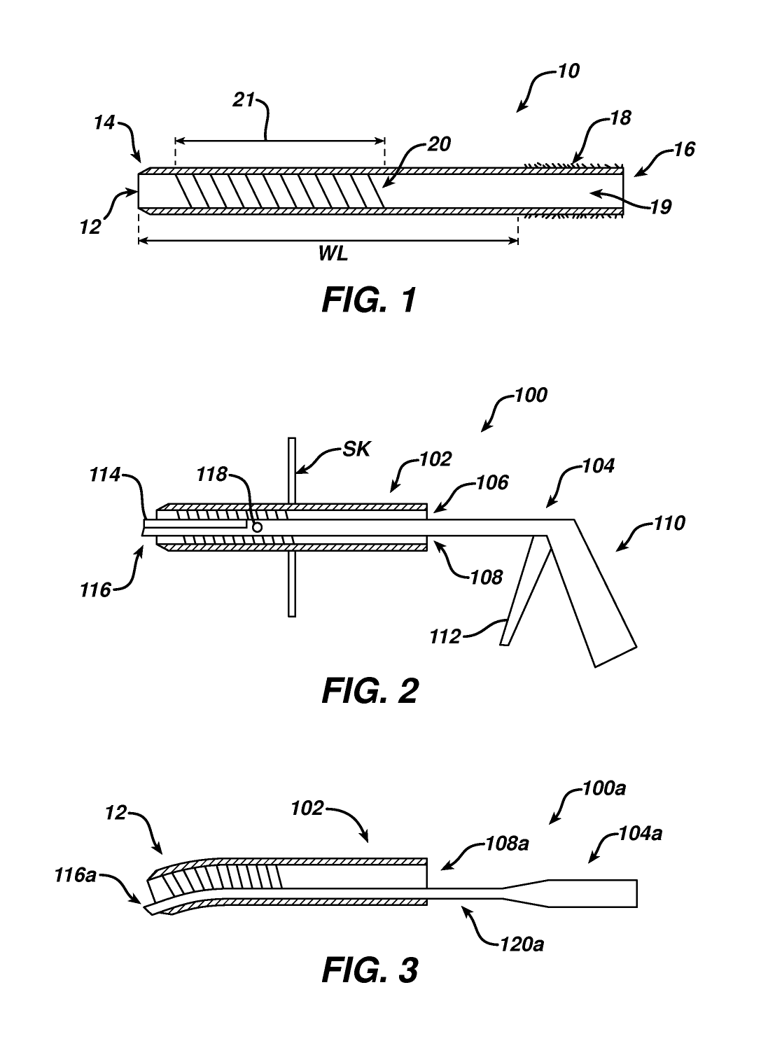

A tissue cutting device according to the present invention is illustrated in FIG. 1 as a cutting tube 10 having a distal end 12 with an annular cutting edge 14 and a proximal end 16 with an enhanced gripping surface 18. Tube 10 defines an open passage 19 and is preferably formed of a biologically compatible metal or metal alloy such as medical grade stainless steel. Cutting edge 14 is ground as a smooth, substantially continuous bevel at distal end 12 in this construction. Gripping surface 18 is a textured, higher-friction pattern that has been grit-blasted or etched about a portion of proximal end 16 in this construction; in other constructions a metallic, ceramic or polymeric material is attached by conventional processes to enhance manual gripping. The term “manual” also includes attachment and manipulation by a robotic arm.

The remaining substantially smooth outer surface of tube 10, distal to gripping surface 18, provides a working length WL which can be inserted through the epidermis of a patient as described in more detail below. In preferred constructions, working length WL is at least three centimeters in length and has an outer diameter of less than three millimeters, more preferably less than or equal to approximately two millimeters. Outer tube diameters less than three millimeters often permit simple bandaging without suturing post-surgery. Working length WL is selected according to the procedure to be performed. For example, knee surgery involving cutting of a meniscus, ligament or tendon or various types of shoulder surgery may require a working length WL of up to four to six inches (approximately 10-15 centimeters).

In this construction, a plurality of cut-outs 20 establish a flexible section 21 and are formed by laser cutting or mechanical grinding in the distal half of tube 10 to enable bending of tube 20 after insertion into a patient, as described in more detail below. In some constructions, cut-outs 20 are aligned in planes perpendicular or transverse to the longitudinal axis of tube 10, and in other constructions the cut-outs 20 are formed in a substantially helical or spiral pattern. Preferably, the cut-outs 20 will not interfere with rotation of tube 20 in at least one of a clockwise and counter-clockwise direction.

A cutting assembly 100 is illustrated in FIG. 2 after cutting tube 102 has been inserted through skin SK of a patient, as described in more detail below. A low-profile grasper 104 has been inserted through proximal end 106 and into open passage 108 of tube 102. Grasper 104 has a handle 110 at its proximal end with lever 112 that operates jaw 114 of grasping element 116 at its distal end. Jaw 114 rotates about pivot 118 when lever 112 is moved proximally. Suitable graspers are commercially available from a number of sources such as East Coast Endoscopy via http://endoscopyforceps.com/UrologyGynecoloyForceps.html. In particular, alligator grasper Part No. 474016 has an outer diameter of 1.6 millimeters and a standard length of forty centimeters, with other lengths and semi-rigid shaft available upon request.

Tube 102 is shown in top view with an alternative curved jaw grasper 104a in FIG. 3. A curved grasper may be desired for some procedures to improve access or to better align the cutting direction with tissue to be removed. If the inner diameter of tube 102 is sufficiently small relative to the outer dimensions of the shaft 120a and grasping element 116a, then insertion of grasper 104a through the distal portion of passage 108a will force tube distal end 12 to bend to confirm to the curvature of grasping element 116a. If the inner diameter of tube 102 is sufficiently large to accommodate curved grasping element 116a without forcing contact against the inners walls of passage 108a, such as illustrated in exaggerated view in FIG. 3, then a surgeon or other operator of grasper 104a may apply lateral pressure to manipulate the curvature of tube distal end 12 after grasper 104 has been inserted.

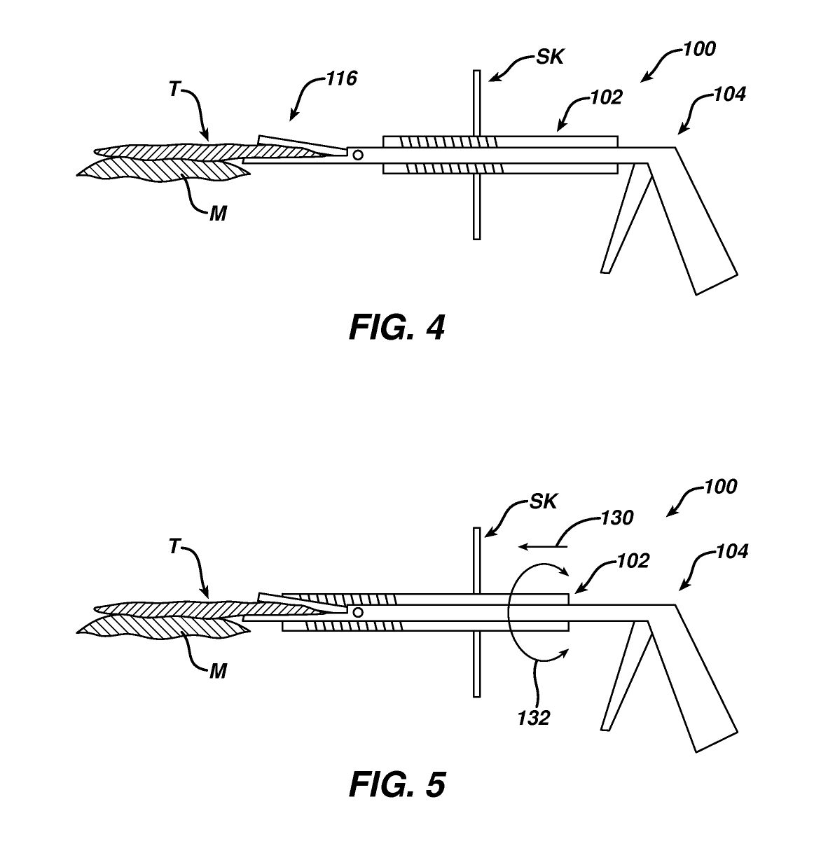

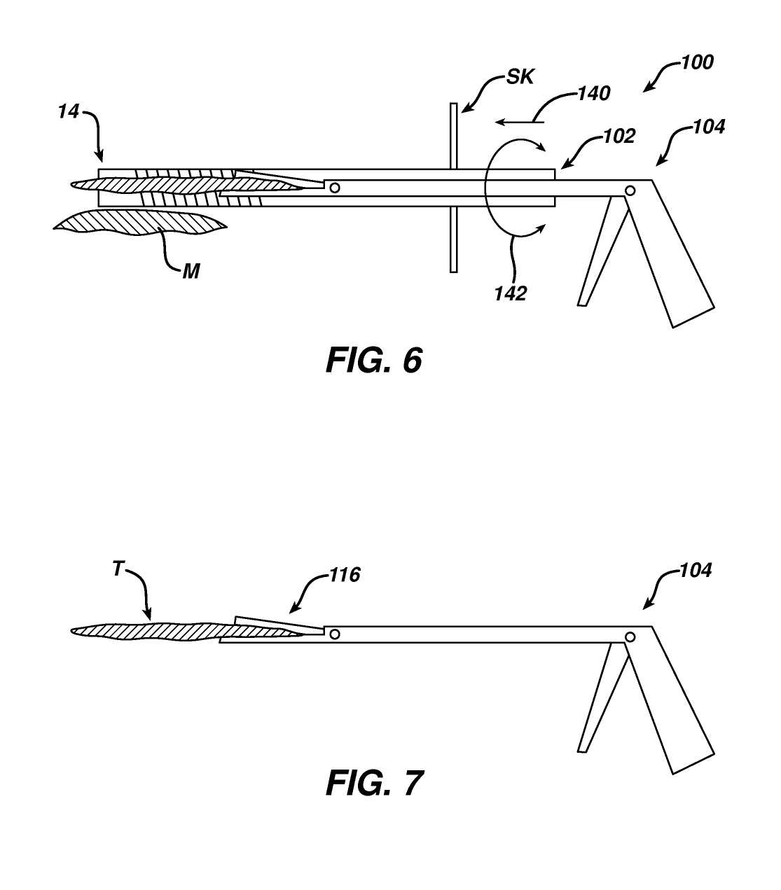

Use of cutting assembly 100, FIG. 2, is illustrated in FIGS. 4-7 to remove a tissue portion T, preferably a non-vascularized tissue such as a ligament, tendon or meniscus, from a tissue mass M, which is the same type of tissue as tissue portion T in some procedures and is a different tissue, such as bone, in other procedures. Grasper 104 is advanced into the patient as shown in FIG. 4 to grasp a portion of tissue T to be cut and removed. After grasping element 116 has been activated to clamp and grasp tissue T, tube 102 is advanced distally as indicated by arrow 130, FIG. 5, until mass M is contacted. Tube 102 is rotated manually in alternating clockwise and counter-clockwise directions as indicated by double-ended arrow 142, FIG. 6, to enhance cutting of tissue around tissue portion T as tube 102 is advanced distally, arrow 140. Tube 102 can also be rotated as shown by double-ended arrow 132, FIG. 5, to assist distal advancement into the patient, arrow 130. After severing tissue T from mass M, grasper 104 is withdrawn proximally to remove tissue T from the patient while it is still clamped in grasping element 116, FIG. 7. The steps illustrated in FIGS. 4-7 are then repeated as needed to remove as much tissue as desired. In yet other constructions, the grasping tool is a flexible wire suture capture device such as a Chia Percpasser available from DePuy Mitek, Inc. of Raynham, Mass.

There are a variety of techniques for introducing a cutting tube according to the present invention into a patient to achieve the position of assembly 100 as illustrated in FIG. 2. For a number of arthroscopic procedures, a conventional spinal needle is inserted into the joint space and saline is injected under pressure to expand the working space around tissue to be cut. An initial insertion site for tube 102 is prepared in some constructions by first inserting a second spinal needle at the site to form an initial channel in the patient, withdrawing the needle, and then widening the channel using an obturator as discussed in more detail below.

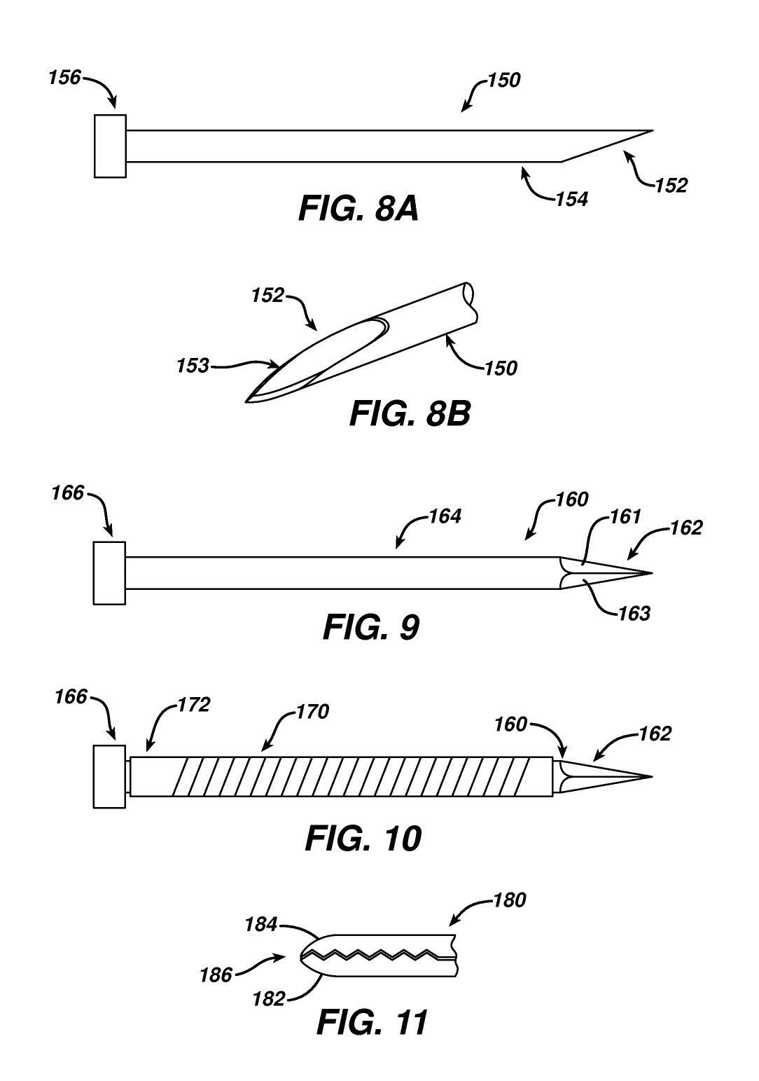

In other constructions, a hollow trocar 150, FIG. 8A, or a solid trocar 160, FIG. 9, is first inserted within the passage of a cutting tube 170, as shown for trocar 160 in FIG. 10. Hollow trocar 150, FIG. 8A, has an elongated shaft 154 defining a central lumen with a distal end 152 which is shown in more detail in FIG. 8B having a triple-ground Quincke tip 153. A non-coring-type tip is preferred to minimize the trauma to tissue in the channel formed in the patient. An enlarged head 156 is attached to or formed on the proximal end of shaft 154 to facilitate driving trocar 150 into the patient. Trocar 150 can also be made solid, without a central lumen, or with an obturator for the lumen.

Similarly, trocar 160, FIG. 9, has a solid trocar tip 162, having a plurality of facets 161 and 163, at the distal end of shaft 164 and a head 166 at the proximal end of shaft 164. During use, trocar 160 is slid into cutting tube 170 so that head 166 abuts the proximal end 172 of tube 170, FIG. 10. Pressure is applied to the combination to drive it into position. The trocar 160 is then removed and a grasping tool inserted and utilized as described above.

For certain arthroscopic procedures, trocar 160 and tube 170 are formed of stainless steel such as SS 304 for single use, or of a harder grade material such as 17-4 hardened SS for reusable devices to maintain a sharp cutting edge. Shaft 164 has an outer diameter that is nearly as large as the inner diameter of tube 170; for example, if tube 170 has an outer diameter of 2.0 millimeters and an inner diameter of 1.7 millimeters, then shaft 164 has an outer diameter of slightly less than or equal to 1.7 millimeters.

Instead of a sharp-tipped trocar, an obturator having similar overall dimensions but with a blunt, atraumatic tip can be utilized, especially if an initial channel has been formed with a shallow scalpel incision or by prior insertion of a needle of 18 gauge or larger diameter. In one construction, the grasping tool itself serves as the obturator. A grasping element 180, FIG. 11, has first and second toothed jaws 182 and 184 which form a rounded distal tip 186 when closed. The outer diameter of grasping element 180 is sized to fit the inner diameter of a cutting tube according to the present invention.

Thus, while there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps that perform substantially the same function, in substantially the same way, to achieve the same results be within the scope of the invention. Substitutions of elements from one described embodiment to another are also fully intended and contemplated. It is also to be understood that the drawings are not necessarily drawn to scale, but that they are merely conceptual in nature. It is the intention, therefore, to be limited only as indicated by the scope of the claims appended hereto.

Every issued patent, pending patent application, publication, journal article, book or any other reference cited herein is each incorporated by reference in their entirety.

What is claimed is:

1. A tissue cutting assembly, comprising:
a generally cylindrical tube having a distal end and a proximal end and defining an open passage extending from the proximal end to the distal end, the distal end defining a cutting edge, and the tube having a substantially smooth exterior along a working length selected for insertion through the epidermis of a patient to access tissue to be cut;
a grasping tool having a distal portion defining a grasping element capable of grasping tissue and of being inserted through the passage of the tube; and
the tube and the grasping tool being separable and independent from each other, the tube being advanceable relative to the tool to cut tissue held by the grasping element.

2. The assembly of claim 1 wherein the cutting edge is substantially annular.

3. The assembly of claim 1 wherein the cutting edge is substantially smooth.

4. The assembly of claim 1 wherein at least a portion of the tube is capable of being bent during insertion of the grasping element.

5. The assembly of claim 4 wherein the tube defines at least one cut-out such that the tube is capable of being bent during insertion of the grasping element.

6. The assembly of claim 1 wherein the proximal portion of the tube includes an enhanced gripping surface.

7. The assembly of claim 1 wherein the working length of the tube has a length of at least three centimeters and has an outer diameter of less than three millimeters.

8. A method of cutting tissue, comprising:
inserting into a patient a generally cylindrical tube having a distal end and a proximal end and defining an open passage extending from the proximal end to the distal end, the distal end defining a cutting edge;
inserting into the passage a grasping tool having a distal portion defining a grasping element capable of grasping tissue and of being inserted through the passage of the tube;
grasping a portion of tissue with the grasping element;
advancing the tube to cut tissue around the grasped portion; and
removing the grasped portion of the tissue from the patient;
wherein the tube and the grasping tool are separate and independent from each other.

9. The method of claim 8 wherein advancing the tube includes rotating it at least during contact with tissue to be cut.

10. The method of claim 9 wherein the tube is rotated manually.

11. The method of claim 8 wherein at least a portion of the grasping element is curved and advancing the tube includes enabling the tube to bend and to be guided by the grasping element.

12. The method of claim 8 wherein the cutting edge is substantially annular.

13. The method of claim 8 wherein the cutting edge is substantially smooth.

14. The method of claim 8 wherein the tube is less than three millimeters in diameter.

15. The method of claim 8 wherein inserting the tube includes placing one of a trocar and an obturator in the passage of the tube to substantially block the passage, pushing the tube through the epidermis and underlying tissues until a desired depth is reached, and then removing the one of the trocar and the obturator.

16. A tissue cutting assembly consisting essentially of:
a generally cylindrical metallic tube having a distal end and a proximal end and defining an open passage extending from the proximal end to the distal end, the passage lacking a seal, and the distal end defining a cutting edge that is substantially continuous about the distal end;
a grasping tool having a distal portion defining a grasping element capable of grasping tissue and of being inserted through the passage of the tube; and
the tube and the grasping tool being separable and independent from each other, the tube being advanceable relative to the tool to cut tissue held by the grasping element.

17. The assembly of claim 16 wherein the tube is less than three millimeters in diameter.

18. The assembly of claim 16 wherein at least a portion of the tube is capable of being bent during insertion of the grasping element.

19. The assembly of claim 17 wherein the tube defines a plurality of cut-outs such that the tube is capable of being bent during insertion of the grasping element.

20. The assembly of claim 16 further including one of a trocar and an obturator sized to substantially block the distal end of the passage during insertion of the tube into a patient.

21. The assembly of claim 16 wherein the proximal portion of the tube includes an enhanced gripping surface.

22. A tissue cutting device consisting essentially of a generally cylindrical metallic tube having a distal end and a proximal end and defining an open passage extending from the proximal end to the distal end, the distal end defining a cutting edge, and the tube having a substantially smooth exterior along a working length selected for insertion through the epidermis of a patient to access tissue to be cut, wherein the working length of the tube has a length of at least three centimeters and has an outer diameter of less than three millimeters.
